(19) United States
(12) Patent Application Publication
(10) Pub. No.: US 2011/0282172 A1
(43) Pub. Date: Nov. 17, 2011

(54) LONG-TERM IMPLANTABLE BIOSENSOR

(75) Inventor: Richard G. Sass, Portland, OR (US)

(73) Assignee: ISENSE CORPORATION, Wilsonville, OR (US)

(21) Appl. No.: 13/127,708

(22) PCT Filed: Nov. 9, 2009
(86) PCT No.: PCT/US2009/063760

§ 371 Date: Jul. 20, 2011

Related U.S. Application Data
(60) Provisional application No. 61/113,486, filed on Nov. 11, 2008.

(51) Int. Cl. A61B 5/1468; A61B 5/145

(52) U.S. Cl. 600/347; 600/345

(57) ABSTRACT

Embodiments provide an analyte sensing device having one or more indicating electrodes adapted for long-term use within an individual. An indicating electrode coupled with a reference electrode may be inserted within or below the dermis of an individual and may be electrically coupled to an external sensor unit. Related analyte sensor insertion aids and methods of using the disclosed embodiments are also provided.

### CROSS REFERENCE TO RELATED APPLICATIONS

The present application claims priority to U.S. Provisional Patent Application No. 61/113,486, filed Nov. 11, 2008, entitled “Long-term Implantable Biosensor,” the entire disclosure of which is hereby incorporated by reference in its entirety.

### TECHNICAL FIELD

Embodiments herein relate to the field of biomedical devices, and, more specifically, to analyte sensors adapted for long-term implantation into a body.

### BACKGROUND

Methods commonly available for the periodic testing of blood glucose levels currently require the diabetic individual to pierce his or her skin for each test. Patients find this process inconvenient and painful, leading to a reduction in compliance with recommended testing. The failure to monitor blood glucose levels is associated with potentially serious consequences. One possible solution to the lack of compliance with testing regimes is to use a semi-implantable sensor designed to be inserted below the skin of the patient. The semi-implantable sensor may be coupled to an external monitor that measures electrical current from the sensor and interprets the data to display a value corresponding to the concentration of an analyte. Examples of such systems are described in U.S. Pat. No. 7,310,544 to Brister et al. and U.S. Pat. No. 5,165,407 to Wilson et al. Other analytes may also be monitored using such systems.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates an exemplary sensing device in accordance with various embodiments;

FIGS. 2a-2d illustrate lower surface features of an analyte sensing device base in accordance with various embodiments;

FIGS. 3a-3f illustrate sensor contact configurations in accordance with various embodiments;

FIGS. 4a-4c illustrate electrode configurations in accordance with various embodiments; and

FIGS. 5a-5b illustrate exemplary analyte sensing devices inductively coupled to a sensor assembly in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the intended scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the scope of embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments herein, are synonymous.

Methods, apparatuses, and systems for monitoring an analyte in an individual are provided. Embodiments provide an analyte sensing device having one or more indicating electrodes. Each indicating electrode may serve as an anode, a cathode, or a combination of anode and cathode. There may be any suitable number of electrodes, such as 1, 2, 3, 4, 5, or more indicating electrodes. Some embodiments may include a reference electrode, an indicating electrode and, optionally, a counter electrode. In some situations, the reference electrode may function as a counter electrode. Additional details pertaining to indicating electrodes may be found in U.S. Pat. Nos. 5,965,380; 5,165,407; 7,146,202; and U.S. patent application Ser. No. 10/640,980, the entire contents and disclosures of which are hereby incorporated by reference.

Various indicating electrodes of a plurality of indicating electrodes may be each configured to detect different analytes, or, alternatively, each indicating electrode may be configured to detect the same analyte, such as lactose, glucose, etc.

One or more indicating electrodes may be provided and may serve as the anode(s), while a base unit configured to be coupled to one or more indicating electrodes may have a skin contact surface that serves as the cathode.

Thus, there is provided an analyte sensing device comprising a base unit configured to reside on the skin of an individual when in use, the base unit having one or more electrical components and one or more downward projecting prongs configured to be retained within the skin of the individual, and an analyte indicating electrode and a reference electrode electronically coupled to the base unit, the electrodes configured for insertion below the epidermis of the individual. The electrodes may be separated or in contact with one another. If the electrodes are in contact, they may be at least partially entwined. For example, the electrodes may be helically entwined along at least a portion of their lengths. The electrodes may be entwined in a regular or irregular helix or one electrode may be wrapped around another electrode. In an embodiment having three electrodes, the electrodes may be braided, twisted, regularly or irregularly entwined, and/or one or two electrodes may be wrapped around a third electrode.

For the purposes of the description, the phrase “helically entwined” means that two, three or more elements are twisted together to form a regular or irregular helix or spiral along some portion of the lengths of those elements. “Helically entwined” may mean that elements are twisted together only along one portion of the lengths of the elements, along more than one portion of the lengths of the elements, or along the entire lengths of the elements.

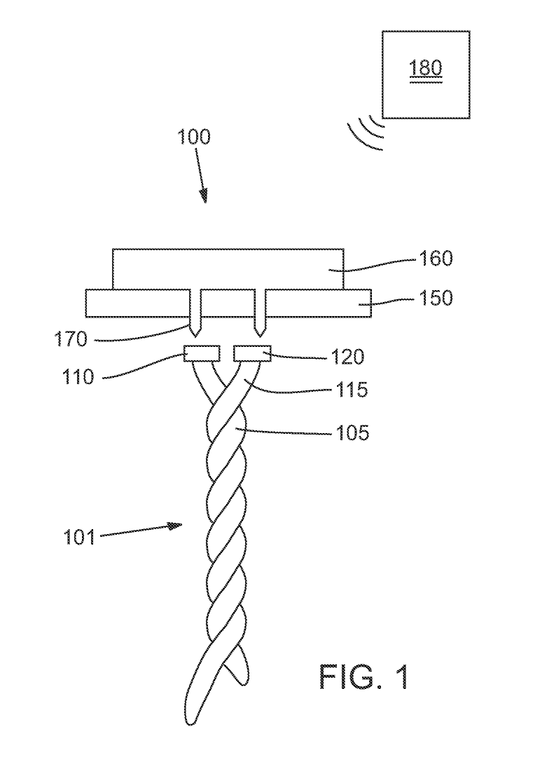

FIG. 1 illustrates an exemplary analyte sensing device 100 with a sensor assembly 101, a patch 150, a base unit 160, base unit contact elements 170, and electronics monitoring unit 180. Sensor assembly 101 further includes an indicating electrode 105, an indicating electrode contact element 110, a reference electrode 115, and a reference electrode contact element 120. Electrodes 105 and 115 are elongated, meaning that they have an extended, longitudinal configuration.

As shown in FIG. 1, indicating electrode 105 and reference electrode 115 are helically entwined along a portion of their lengths. Indicating electrode contact element 110 may be electrically coupled to indicating electrode 105, and reference electrode contact element 120 may be electrically coupled to reference electrode 115. Patch 150 may be coupled to the lower surface of base unit 160 using an adhesive and/or one or more mechanical coupling elements. Base unit contact elements 170 may project downward from the lower surface of base unit 160 through patch 150, and may be separate/interchangeable components of base unit 160/patch 150 or may be integral components of base unit 160/patch 150. In use, one or more base unit contact elements 170 may be electrically coupled to indicating electrode contact element 110, while one or more other base unit contact elements 170 may be electrically coupled to reference electrode contact element 120. Electronics monitoring unit 180 may be electromagnetically coupled to or otherwise in communication with base unit 160.

In operation, sensor assembly 101 may be contained within a trocar that is inserted through the skin of an individual. Sensor assembly 101 may be ejected from the trocar into the tissues below the epidermis, with indicating electrode contact element 110 and reference electrode contact element 120 retained above, within or below the dermis. Indicating electrode 105 and reference electrode 115 may be retained below the dermis and may be in contact with sub-dermal fluids/analytes. Sensor assembly 101 may be inserted into/through the skin of an individual without the use of a trocar, the sensor assembly 101 being inserted manually or with the aid of an insertion device. The sensor assembly may be inserted by the user or by a physician, a nurse, a technician, an assistant, etc. The sensor assembly may be inserted surgically.

The bottom surface of patch 150 may be placed against the epidermis of the individual over the insertion site, such that base unit contact elements 170 project downward through the epidermis, whether through a previously formed opening, incision or insertion site or through newly formed insertion paths. Base unit contact elements 170 may be coupled to indicating electrode contact element 110 and/or reference electrode contact element 115.

Electrochemical reactions or detection of various charged species occurring at indicating electrode 105 may generate an electrical current that may flow through indicating electrode 105 to indicating electrode contact element 110, from indicating electrode contact element 110 to one or more base unit contact elements 170 coupled to indicating electrode contact element 110, and from one or more base unit contact elements 170 to other portions of base unit 160. Base unit 160 may sense the electrical current and may transmit information through a wired or wireless connection to electronics monitoring unit 180. Electronics monitoring unit 180 may analyze, display, transmit, and/or store the transmitted information.

Indicating electrode 105 may be an anode and reference electrode 115 may be a cathode. Any suitable materials or arrangements may be used to form electrodes 105 and 115, whether constructed from one or more materials, coatings, etc. Electrodes may be flexible, semi-rigid or rigid and may be flat, round, square, etc. For example, one or more electrodes may include a wire that is rigid, semi-rigid or flexible. Indicating electrode 105 may comprise a flexible core, upon the surface of which may be disposed an electrochemically active metal. Reference electrode 115 may comprise a flexible core, upon the surface of which may be disposed such a metal. Metals disposed upon the surface of indicating electrode 105 may include, but are not limited to, platinum, palladium or gold, and metals disposed upon the surface of reference electrode 115 may include, but are not limited to, silver or silver chloride. An electrochemically active metal may contact the core and may surround the core. The term “surround” as used herein means that the electrochemically metal completely covers/encases the core, not that it simply encircles the core, such that the core is shielded from contact by fluids, etc. that are external to the electrode. The flexible core may be constructed of tantalum, glass fiber, expanded polytetrafluoroethylene, silicone, or other flexible material, and may be flat, rectangular, cylindrical, or have any other suitable shape.

An electrochemically active metal such as platinum, palladium or gold may be deposited on the surface of the flexible core by vacuum deposition, electroless deposition, sputtering, ion plating, arc spraying, plasma spray coating, chemical vapor deposition, molecular beam epitaxy, sol gel processing, spin coating, pulsed laster deposition, or any other method. Indicating electrode 105 may be coated along a portion of its length by a layer of dielectric (not shown), such as a layer comprising polyimide. One or more portions of the dielectric layer may be removed to expose the underlying metal. A dielectric layer or spacer may be used to electrically separate indicating electrode 105 from reference electrode 115.

Indicating electrode 105 may be at least partially covered with one or more membranes to regulate contact between or among analytes, oxygen, elements, compounds, hydrogen peroxide, and/or any other component of blood or interstitial fluid and the metal disposed upon the surface of indicating electrode 105. Enzymes and/or an enzyme system or membrane may also be disposed upon the surface of one or more membranes or deposited between membranes. An interferent reducing inner layer, an enzyme layer (for example including glucose oxidase, lactate oxidase and/or lactate dehydrogenase), and a selectively permeable outer membrane layer may be disposed upon the surface of an electrode. A silane layer may be provided under and/or over the enzyme layer. Exemplary suitable membranes and enzymes may be found in U.S. Pat. Nos. 5,165,407 and 6,613,379, and in U.S. patent application Ser. No. 11/538,340, the entire disclosures of which are hereby incorporated by reference. Membranes and/or enzymes may be applied to the surface of indicating electrode 105 over one or more layers of metal and/or over a dielectric. One or more portions of a dielectric may be removed from indicating electrode 105 prior to the addition of membranes and/or enzymes.

Indicating electrode 105 and reference electrode 115 may be constructed separately and subsequently coupled and/or entwined along their lengths. Alternatively, indicating electrode 105 and reference electrode 115 may be constructed together such that subsequent coupling is not necessary. For example, a single core may be formed and two metals may be sequentially disposed along separate surfaces of the core and/or disposed along the core in a pattern such as in separate spirals. In some embodiments, two metals may be disposed along a single core to form entwined electrodes on one substrate (e.g. disposed in spirals, parallel lines, etc.).

When an indicating electrode is coupled/entwined with a reference electrode, the coupled/entwined electrodes may be severed at intervals of 10-15 millimeters to create one, two, three or a plurality of coupled sensors 10-15 millimeters in length. In some embodiments, entwined sensors may be 1-5 millimeters in lengths, while, in others, entwined sensors may be 5-10 millimeters in length, 15-20 millimeters in length, or 20-30 millimeters, or more than 30 millimeters in length.

An electrode may be coupled to another electrode using an adhesive, ultrasonic bonding, physical manipulation, or another suitable technique. For example, electrodes may be coupled by twisting the electrodes together. Electrodes may also be coupled by wrapping one electrode around another, by tying the electrodes together, by wrapping a coupling element around two, three or more electrodes, by dip-coating two or more electrodes together along some portion of their lengths with a suitable material, etc. Coupled electrodes may be blunt or cut on a bias to provide a pointed end and/or may be capped at one or more ends.

Indicating electrode 105 may comprise a relatively flat, rectangular, expanded-polytetrafluoroethylene core, with platinum disposed upon the surface of the core by vacuum deposition to a thickness of approximately 200 microns, and reference electrode 115 may comprise a flat, rectangular, expanded-polytetrafluoroethylene core, with silver disposed upon the surface of the core to a thickness of approximately 200 microns. Other embodiments may vary in composition and may include metals disposed upon the surface of a rigid, flexible, or semi-rigid core by other methods. One or both electrodes may be coated with polyimide, and portions of the polyimide may be subsequently removed as desired.

Indicating electrode contact element 110 may be constructed of or coated with a metal and may be mechanically coupled to indicating electrode 105. The metal of indicating electrode contact element 110 may be the same or a different metal used for or coated on indicating electrode 105. Indicating electrode contact element 110 may be coupled to indicating electrode 105 with a hinge, a pin, a pivot, a swivel, or other connector mechanism that allows for movement of indicating electrode contact element 110 in at least one plane relative to indicating electrode 105. One end of indicating electrode 105 may be inserted through some portion of indicating electrode contact element 110. Indicating electrode contact element 110 may be removable from indicating electrode 105.

Indicating electrode contact element 110 may include a transmitter that communicates with base unit 160 and/or with electronics monitoring unit 180. Indicating electrode contact element 110 may include a memory that retains information until it is communicated to base unit 160 and/or to electronics monitoring unit 180.

Indicating electrode contact element 110 may include a projection that protrudes upward through the epidermis to contact base unit 160 and/or base unit contact elements 170. Indicating electrode contact element 110 may include an element such as a string, band, wire, hook, loop, etc. to facilitate the removal of indicating electrode contact element 110 and/or sensor assembly 101. Sensor assembly 101 may be designed to remain implanted below the epidermis of an individual for 3, 4, 5, 6, or more months, or even up to one year or more.

Reference electrode contact element 120 may be constructed of or coated with a metal and may be coupled to reference electrode 115. The metal of reference electrode contact element 120 may be the same or a different metal used for or coated on reference electrode 115. Reference electrode contact element 120 may be coupled to reference electrode 115 with a hinge, a pin, a pivot, a swivel, or other connector mechanism that allows for movement of reference electrode contact element 120 in at least one plane relative to reference electrode 115. One end of reference electrode 115 may be inserted through some portion of reference electrode contact element 120. In embodiments, reference electrode contact element 120 may be removable from reference electrode 115.

Reference electrode contact element 120 may include a transmitter that communicates with base unit 160 and/or with electronics monitoring unit 180. Reference electrode contact element 120 may include a memory that retains information until it is communicated to base unit 160 and/or to electronics monitoring unit 180.

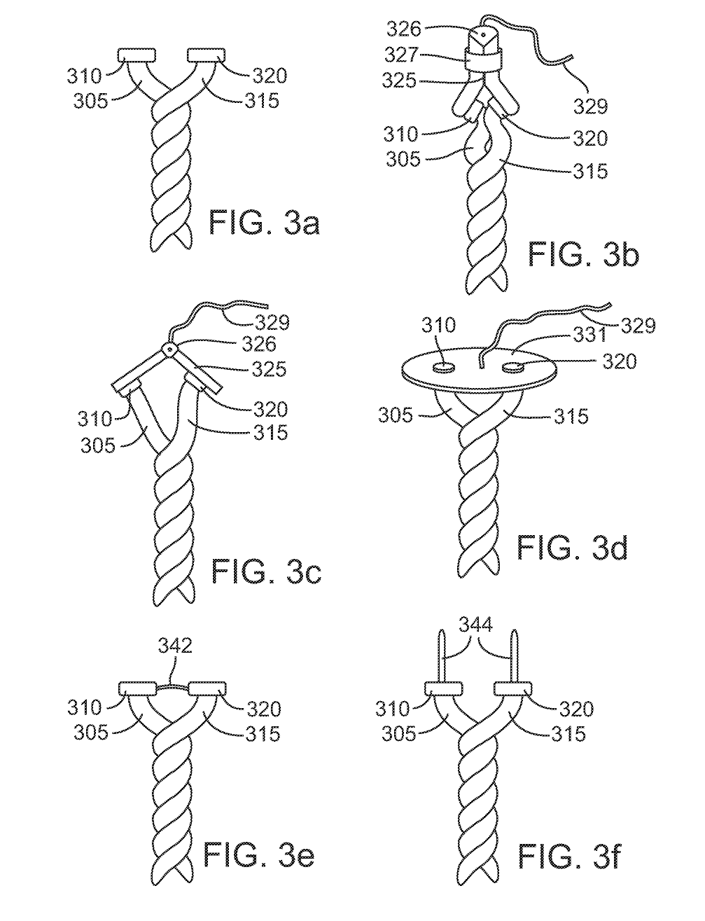

Reference electrode contact element 120 may include a projection that protrudes upward through the epidermis to contact base unit 160 and/or base unit contact elements 170. Reference electrode contact element 120 may include an element such as a string, band, wire, hook, loop, etc., one end of which may remain on the surface of the skin to facilitate the removal of indicating electrode contact element 110 and/or sensor assembly 101. Reference electrode contact element 120 and/or indicating electrode contact element 110 may be coupled to an element that maintains distance between the contact elements and/or facilitates retraction of the contact elements through the dermis and epidermis (see FIGS. 3b-3d).

In an exemplary embodiment, patch 150 may include on its bottom surface a liquid, gel, solid or semisolid adhesive adapted to adhere removably to a mammalian epidermis, and may include on its upper surface a liquid, gel, solid or semisolid adhesive adapted to adhere removably to the lower surface of base unit 160. Patch 150 may include an adhesive that is dissolvable and/or is absorbed over some period of time by mammalian skin. Patch 150 may be affixed to mammalian skin with an adhesive that is not dissolvable or absorbable. Alternatively, patch 150 may not include a pre-applied adhesive, but rather an adhesive may be applied separately. Patch 150 may also include an antimicrobial substance such as an antibiotic medication, a metal, etc. Patch 150 may be elastic, flexible and/or gas permeable. Patch 150 may be water resistant, water repellent and/or may cover some portion of base unit 160.

Base unit 160 may be a single unit, or may include two, three, four or more units. Base unit 160 may include a memory component, a battery component, a transmitter, a receiver, a transceiver, a processor, a solar panel, and/or a display component, etc., any or all of which may be housed/coupled within/on base unit 160. Base unit 160 may be powered by a battery, or may be powered by solar energy, movement, or another energy source. Some embodiments may be constructed without a patch 150, and base unit 160 may be adhered directly to the skin of an individual with an adhesive. Electronics monitoring unit 180 may replace some or all functions of base unit 160. Thus embodiments may be constructed without a base unit 160; and therefore electronics monitoring unit 180 may include a memory component, a battery component, a transmitter, a receiver, a transceiver, a processor, and/or a display component, etc., any or all of which may be housed/coupled within/on electronics monitoring unit 180.

Electronics monitoring unit 180 may be in electrical/electromagnetic communication with and/or may inductively coupled with sensor assembly 101. Base unit contact elements 170 of base unit 160 may be physically connected to sensor assembly 101. Alternatively, base unit 160 may be constructed without base unit contact elements 170 and/or may be electrically coupled to sensor assembly 101. Base unit 160 may be inductively/electromagnetically coupled to sensor assembly 101 (see FIG. 5a).

Base unit 160 may be circular, rectangular, square, or have any other suitable shape, and may be rigid or at least partially flexible. Base unit 160 may be incorporated into a flexible patch and may include an adhesive on a bottom surface. Alternatively, base unit 160 may be worn by an individual on an elastic/adjustable strap, a bracelet, a wrap, etc.

Electronics monitoring unit 180 may be a battery-powered unit designed to be worn/carried by an individual. Electronics monitoring unit 180 may be rechargeable. Electronics monitoring unit 180 may be powered and/or recharged by the motion of the individual wearing/carrying the unit, by solar energy, or another energy source.

An insertion device such as a trocar and/or syringe may be provided for insertion of sensor assembly 101 beneath an epidermis. An insertion device may accommodate a sensor assembly 101 within an inner cavity. An insertion device may include a plunger or other mechanism to mechanically push sensor assembly 101 from the interior of the insertion device into/below/between one or more tissues of an individual. Sensor assembly 101 may be ejected from the interior of the insertion device by fluid, air pressure, manual force, a spring release element, etc. An insertion device may be a hollow trocar, and sensor assembly 101 may be manually inserted into the tissues of an individual through the interior cavity of the trocar after piercing the skin with the trocar.

An insertion device may comprise one or more parts whether affixed to base unit 160, removable from base unit 160, or otherwise coupled to base unit 160. In some embodiments an insertion device may not be coupled to base unit 160.

Some portion of a sheath/catheter disposed on the exterior of an insertion device may remain at least partially within the tissues of the individual after insertion of sensor assembly 101. A thin elongated element such as a sheath, catheter, band, string, etc. may be disposed on the exterior surface or within an interior cavity of an insertion device and may remain attached at one end to sensor assembly 101 after insertion, with the other end of the thin elongated element remaining on the surface of the individual's skin at the insertion site to facilitate removal of sensor assembly 101. A thin elongated element may be constructed of a polymer, silicone, expanded polytetrafluoroethylene, or other biocompatible material. A thin elongated element comprising an electrically conductive material such as metal may be connected to indicating electrode contact element 110, and a separate thin elongated element comprising an electrically conductive material such as metal may be connected to reference electrode contact element 120, with both thin elongated elements mechanically coupled to base unit 160. A thin elongated element may be coated with a dielectric, an insulating material, and/or an antimicrobial substance such as a metal, an antibiotic compound, etc.

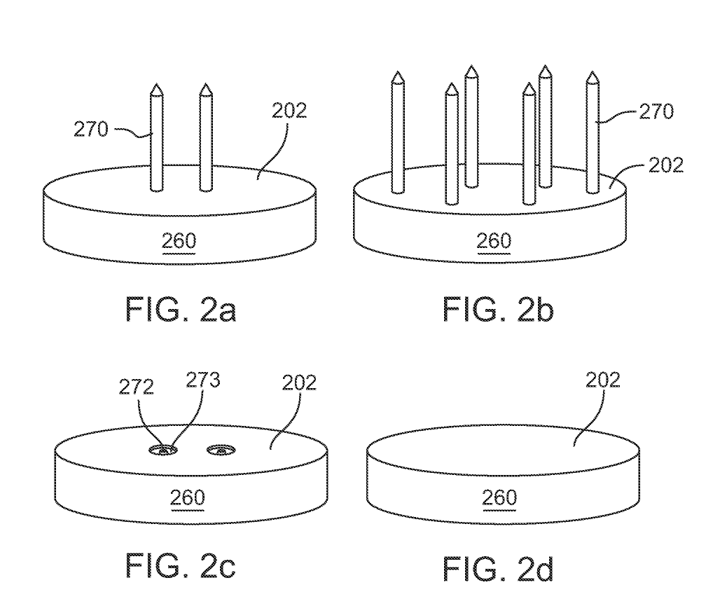

FIGS. 2a-2d illustrate lower surface features of an analyte sensing device base in accordance with various embodiments. Base unit contact elements 270 may vary in shape, size, and/or number, and variations may include but are not limited to those illustrated. FIG. 2a illustrates a lower surface 202 of an exemplary base housing unit 260, which may include two base unit contact elements 270. In operation, after a sensor assembly is inserted within the tissues of an individual, base unit 260 may be placed against the skin of the individual and positioned such that base unit contact elements 270 pierce the skin of the individual over the insertion site. One or more base unit contact elements 270 may contact the implanted indicating electrode contact element, and another one or more base unit contact elements 270 may contact the implanted reference electrode contact element. Base unit contact elements 270 may remain in place within the skin of an individual until the sensor assembly is removed. Base unit contact elements 270 may be adapted to remain within the skin of an individual for a period of days, weeks, or months, such as 1, 2, 3, 4, 5, or 6 months. Base unit contact elements 270 may be removable from base unit 260. This may be done to facilitate removal of a sensor assembly and/or an electrode, an electrode sensor contact element, etc. Base unit contact elements 270 may be periodically replaced with new base unit contact elements 270. Base unit contact elements 270 may be threaded and may be reversibly coupled to base unit 260. Base unit contact elements 270 may be retained within base unit 260 and may be rapidly extended using a motive force sufficient to penetrate skin.

FIG. 2b illustrates a lower surface 202 of a base unit 260 and includes two base unit contact elements 270. In operation, this embodiment is similar to that illustrated in FIG. 2a, but with six base unit contact elements 270. In other embodiments, there may be 1, 2, 3, 4, 5, 6, 7, 8, 9, 10, or more base unit contact elements 270.

FIG. 2c illustrates a lower surface 202 of a base unit 260 and includes two alternative base unit contact elements 272 disposed within lower surface concavities 273. An electrically conductive thin elongated element may be coupled to a reference electrode contact element and a separate electrically conductive thin elongated element may be coupled to an indicating electrode contact element, with each thin elongated element coupled to an alternative base unit contact element 272. An electrically conductive thin elongated element may be coated with a dielectric, an insulating material, and/or an antimicrobial substance such as a metal, an antibiotic compound, etc. Alternative base unit contact elements 272 may be removable from base unit 260 to allow removal of base unit 260 without altering the position of the coupled sensor assembly. Alternative base unit contact elements 272 may be positioned on/within base unit 260.

FIG. 2d illustrates a lower surface 202 of a base unit 260. The illustrated embodiment may be constructed without a base unit contact element 270 and/or an alternative base unit contact element 272. Base unit 260 may be electrically/electromagnetically/inductively coupled to a sensor assembly having a transmitter/memory.

FIGS. 3a-3f illustrate sensor assembly configurations. Embodiments of a sensor assembly illustrated in FIGS. 3a-3f may include an indicating electrode 305, an indicating electrode contact element 310, a reference electrode 315, and a reference electrode contact element 320. Indicating electrode contact element 310 may be coupled to indicating electrode 305 and reference electrode 320 may be coupled to reference electrode contact element 320. Reference electrode 320 and indicating electrode 310 may be entwined along at least a portion of their lengths. Indicating electrode 305 and reference electrode 315 may be constructed separately and subsequently coupled, and/or may be constructed integrally. Coupled electrodes may be separate at one or both ends or may be closely aligned. Coupled electrodes may be severed at one end such that the coupled electrodes terminate in a point, or may be severed at one end such that the coupled electrodes have a blunt or flat surface at one or both ends. Electrodes may be severed by cutting, crimping, or by other suitable methods. Proximal and/or distal ends of coupled electrodes may be pointed, flat, fused together or splayed apart, etc.

FIG. 3a illustrates an exemplary sensor assembly. A sensor assembly may comprise an indicating electrode 305, an indicating electrode contact element 310, a reference electrode 315, and/or a reference electrode contact element 320. In operation, the sensor assembly may be loaded into a hollow interior chamber of an insertion device such as a trocar and inserted into the tissues of an individual, with reference electrode 315 and indicating electrode 350 placed within or below the dermis. An insulating layer may be disposed upon the surface of indicating electrode contact element 310 and/or reference electrode contact element 320 to prevent electrical communication directly between the two elements. Electrical communication between indicating electrode contact element 310 and reference electrode contact element 320 may be desirable. A sensor assembly may further include a conductive bridge between those elements.

FIG. 3b illustrates a sensor assembly that folds for insertion/removal. FIG. 3b includes an indicating electrode 305, an indicating electrode contact element 310, a reference electrode 315, a reference electrode contact element 320, a hinged element 325, a hinge 326, a receptacle 327, and a filament 329. Hinged element 325 may be at least partially folded at hinge 326 and retained partially within receptacle 327 prior to insertion into an individual, with filament 329 connected to hinged element 325 at/near hinge 326. Indicating electrode contact element 310 may be disposed upon one surface of hinged element 325, while reference electrode contact element may be disposed upon another surface of hinged element 325. Filament 329 may be retained within/along the outer surface of an insertion device such that one end of filament 329 is accessible on the surface of the skin of an individual after insertion of the sensor assembly. Pressure applied to the sensor assembly at hinge 326 may push hinged element 325 at least partially through receptacle 327, causing hinged element 325 to become laterally extended in two directions from the lower portion of receptacle 327. This extension places reference electrode contact element 320 and indicating electrode contact element 310 on opposite ends of the sensor assembly and anchors the sensor assembly above/within/below the dermis.

Removal of the sensor assembly may be accomplished by grasping filament 329 above the surface of the skin and pulling upward, causing hinged element 325 to bend at hinge 326 and retracting hinged element 325 at least partially into receptacle 327 before the sensor assembly is pulled from the skin. Hinged element 325 and/or hinge 326 may be spring-loaded. Indicating electrode contact element 310 and reference electrode contact element 320 may be integrated into hinged element 325 such that the top and/or bottom surface of each contact element is exposed to facilitate contact with a base unit. Hinged element 325 may include one or more apertures adapted to accommodate a base unit contact element. A wire or other conductive element may physically connect one or both contact elements to a base unit.

FIG. 3c shows a sensor assembly. Reference electrode contact element 320 may be disposed on one portion of a hinged element 325 while indicating electrode contact element 310 may be disposed on another portion of a hinged element 325. The placement of electrode contact element 320 and indicating electrode contact element 310 may be staggered (see FIG. 3b) such that when hinged element 325 folds at hinge 326, the contact elements may be retained within the fold and not in physical contact with one another, allowing portions of hinged element 325 to fold more closely together. Filament 329 may be connected to hinged element 325 at/near hinge 326. Some portion of filament 329 may be accessible at the surface of the skin after insertion of the sensor assembly. Hinge 326 and/or hinged element 325 may be spring-loaded. Hinged element 325 may be in a folded position prior to insertion and may be at least partially unfolded during/after insertion above/within/below the dermis of an individual. Pulling filament 329 may cause hinged element 325 to fold, facilitating the removal of the sensor assembly.

FIG. 3d illustrates a sensor assembly including indicating electrode 305, indicating electrode contact element 310, reference electrode 315, reference electrode contact element 320, and base element 331. Reference electrode contact element 320, indicating electrode contact element 310 and filament 329 may be disposed on the surface of, or integrated within, base element 331. Base element 331 may be constructed of a thin, flexible material that is biocompatible, including but not limited to a polymer, silicone, expanded polytetrafluoroethylene, etc. In operation, base element 331 may be compressed/rolled/folded for placement above/within/below the dermis of an individual. Base element 331 may maintain a separation between reference electrode contact element 320 and indicating electrode contact element 310. A filament, tab, handle, or other graspable element may be coupled to the sensor assembly and may be used to pull the sensor assembly from the skin.

FIG. 3e illustrates a sensor assembly in which a connector/separator 342 may be placed in contact with indicating electrode contact element 310 and/or reference electrode contact element 320. Connector 342 may or may not be conductive. Connector 342 may be used to maintain a physical separation between indicating electrode contact element 310 and reference electrode contact element 320.

FIG. 3f illustrates a sensor assembly in which a prong element 344 may extend from or be disposed upon a surface of indicating electrode contact element 310 and/or upon a surface of reference electrode contact element 320. Prong elements 320 may project upward through the dermis/epidermis of an individual to contact a base unit. Prong elements 320 may function as antennae/transmitters/receivers for indicating electrode contact element 410 and/or reference electrode contact element 320 and may vary in shape, length and/or configuration.

In some embodiments, one or more sensor contacts may be connected to a rigid/semi-rigid/flexible sensor contact element platform that anchors a sensor contact at a desired location above, within or below the dermis. One purpose of a sensor contact element platform may be to separate two sensor contact elements such that there is no physical contact between them. One or more portions of a sensor contact element platform may be designed to be released/break-away from a sensor assembly during/after use of the sensor assembly to facilitate removal of the device.

Some or all of a sensor contact element platform may include material that is absorbed by the mammalian body after implantation; the absorbable portion may be constructed using a polymer that comprises one or more of glycolic acid, lactic acid, para-dioxanone, trimethylene carbonate, caprolactone, polyhydroxyalkanoates, or other biocompatible materials. The absorbable portion may include an alginate, a sugar, a starch, a gelatin, cellulose, polyvinyl alcohol, chitosan, or other biocompatible materials. A sensor contact element platform may be designed to dissolve within a mammalian body within 5, 10, 15, 20, 30 or 60 minutes post implantation, or a sensor contact element platform may be designed to be absorbed more slowly, such as within 1, 2, 3, 4, 5, 6, 7, or 8 weeks. The sensor contact element platform may be designed to be absorbed over a period of time greater than 8 weeks, such as 3, 4, 5, 6 months or more. Alternatively, the sensor contact element platform may be designed to not be absorbed.

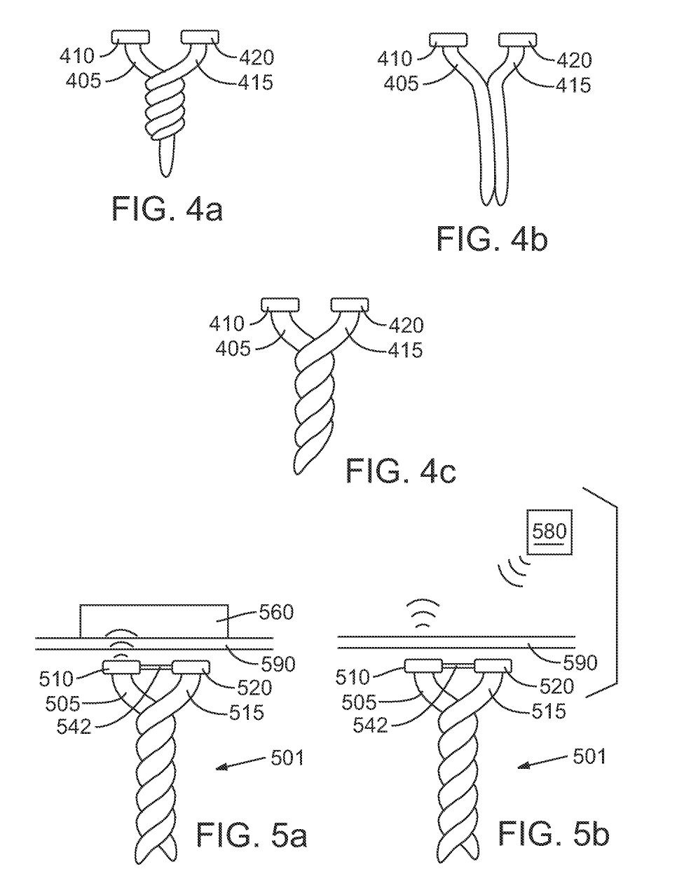

FIGS. 4a-4c illustrate various electrode configurations. Each of the illustrated indicating electrode configurations may include an indicating electrode 405, an indicating electrode contact element 410, a reference electrode 415, and a reference electrode contact element 420. Indicating electrode 405 may be coupled to indicating electrode contact element 410, and reference electrode 415 may be coupled to reference electrode contact element 420. FIG. 4a illustrates an electrode configuration in which reference electrode 415 may be spirally disposed along the exterior of indicating electrode 405. Indicating electrode 405 may be spirally disposed along the exterior of reference electrode 415. An analyte-permeable element or non-permeable element may at least partially wrap/encompass both indicating electrode 405 and reference electrode 415. FIG. 4b illustrates an electrode configuration in which reference electrode 415 and indicating electrode 405 may be coupled along at least a portion of their lengths. FIG. 4c illustrates a similar electrode configuration in which coupled reference electrode 415 and/or indicating electrode 405 may be cut diagonally to produce a pointed end. Electrodes may also be individually severed/cut, fused at one end, cut to produce a flat/curved/pointed end, etc. Reference electrode 415 and indicating electrode 405 may be coupled by an adhesive, by ultrasonic bonding, by twisting/wrapping the electrodes together, etc.

FIGS. 5a-5b illustrate exemplary analyte sensing devices inductively coupled to a sensor assembly 501. FIG. 5a illustrates a base unit 560 that may be inductively coupled to a sensor assembly 501. FIG. 5b illustrates an electronics monitoring unit 580 that may be inductively coupled to a sensor assembly 501. As shown in FIGS. 5a and 5b, a sensor assembly 501 may include an indicating electrode 505, an indicating electrode contact element 510, a reference electrode 515, a reference electrode contact element 520, and a connector/separator 542. Indicating electrode contact element 510 may further include one or more electrical components such as a memory component, a battery component, a transmitter, a receiver, a transceiver, and/or a processor.

In operation, a sensor assembly 501 may be inserted in a variety of locations above, within or below the epidermis, dermis, and/or subdermis of an individual, represented in FIGS. 5a and 5b as dermal layer 590. Indicating electrode contact element 510 and reference electrode contact element 520 may be retained above the epidermis, within the epidermis, below the epidermis, above the dermis, within the dermis, and/or within or below the subdermis. Sensor assembly 501 may be inserted in any suitable place on the body of an individual, such as an arm, a leg, an abdomen, a chest, a back, etc. Electrochemical reactions occurring at reference electrode 505 may generate an electrical current that flows to indicating electrode contact element 510. Indicating electrode contact element 510 may send a varying electromagnetic signal upward through the skin of the patient in response to the current. The varying electromagnetic signal may be received by a base unit 560 (see FIG. 5a) and/or by an electronics monitoring unit 580 (see FIG. 5b). A base unit 560 and/or an electronics monitoring unit 580 may remain above dermal layer 590 and may be retained against dermal layer 590 by an adhesive, a patch, a strap, and/or any other suitable means. Base unit 560 and/or electronics monitoring unit 580 may display information in response to an electromagnetic signal received from sensor assembly 501. A base unit 560 and/or an electronics monitoring unit 580 may send an electromagnetic signal that is received by some portion of a sensor assembly 501. Base unit 560 and/or electronics monitoring unit 580 may include a memory component, a battery component, a transmitter, a receiver, a transceiver, a processor, and/or a display component, a display, etc.

Thus, there is provided an analyte sensing device comprising a sensor assembly inserted below the epidermis and in communication with an ex vivo analyte sensor, the sensor assembly having an indicating electrode, an indicating electrode contact element, a reference electrode, and a reference electrode contact element. Such sensing electrodes may be formed by any suitable process including machining and deposition/coating processes, chemical etching or other micromechanical processes.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. An analyte sensing assembly, comprising:
an elongated indicating electrode configured for insertion below a mammalian epidermis, wherein the indicating electrode comprises a first core and a first layer of electrochemically active metal surrounding and in contact with an outer surface of the core; and
an elongated reference electrode configured for insertion below a mammalian epidermis, wherein the reference electrode comprises a second core and a second layer of electrochemically active metal surrounding and in contact with an outer surface of the core,
wherein the indicating electrode and the reference electrode are coupled along at least a portion of their lengths.

2. The analyte sensing assembly of claim 1, wherein the indicating electrode and the reference electrode are entwined along at least a portion of their lengths.

3. The analyte sensing assembly of claim 1, wherein the indicating electrode and the reference electrode are helically entwined along at least a portion of their lengths.

4. The analyte sensing assembly of claim 1, wherein the first and second core each comprise at least one of silicone and expanded polytetrafluoroethylene.

5. The analyte sensing assembly of claim 1, wherein the first layer of electrochemically active metal is configured to provide at least one sensing surface.

6. The analyte sensing assembly of claim 1, wherein the first layer of electrochemically active metal is at least partially surrounded by a membrane system.

7. The analyte sensing assembly of claim 6, wherein the membrane system is configured to sense glucose.

8. The analyte sensing assembly of claim 3, further including a first sensor contact element coupled to the first layer of electrochemically active metal.

9. The analyte sensing assembly of claim 8, further including a second sensor contact element coupled to the second layer of electrochemically active metal.

10. The analyte sensing assembly of claim 9, wherein the first sensor contact and the second sensor contact are configured to be retained beneath a mammalian epidermis.

11. An analyte sensing system, comprising:
an analyte sensor assembly configured to be retained beneath an epidermis of a body when in use, comprising an indicating electrode and a reference electrode, wherein the indicating electrode and the reference electrode are helically entwined along some portion of their lengths; and
an analyte sensor module configured to removably couple with the analyte sensor assembly.

12. The analyte sensing system of claim 11, wherein the analyte sensor module is configured to be mounted on the outer surface of the skin of an individual.

13. The analyte sensing system of claim 11, further including a first sensor contact coupled to the indicating electrode and adapted to pivot in relation to a point of attachment to the indicating electrode, and a second sensor contact coupled to the reference electrode and adapted to pivot in relation to a point of attachment to the reference electrode.

14. The analyte sensing system of claim 11, wherein the analyte sensor module comprises an adhesive patch.

15. The analyte sensing system of claim 11, wherein the analyte sensor module is configured to inductively couple with the analyte sensor assembly.

16. The analyte sensing system of claim 11, wherein the analyte sensor module further includes along a bottom surface at least one downward projection configured to be retained at least partially within the epidermis.

17. The analyte sensing system of claim 11, further including a first sensor contact coupled to the indicating electrode, and a second sensor contact coupled to the reference electrode.

18. The analyte sensing system of claim 17, wherein the analyte sensor module further includes along a bottom surface at least one downward projection configured to be retained at least partially within the epidermis, wherein each of the at least one downward projection is coupled to either the first sensor contact or the second sensor contact.

19. The analyte sensing system of claim 11 wherein the analyte sensor module is configured to receive electromagnetic signals from the analyte sensor assembly.

20. The analyte sensing system of claim 11 wherein the analyte sensor module is inductively coupled to the analyte sensor assembly.

21. A method for monitoring an analyte within a body having an epidermis, comprising:
inserting within a body an analyte sensing element including an elongated indicating electrode surrounded at least partially by an insulating layer, and an elongated reference electrode, wherein the indicating electrode and the reference electrode are entwined along at least a portion of their lengths;
placing a sensor module above the epidermis of the body in proximity to the analyte sensing element;
coupling the analyte sensing element and the sensor module; and
receiving by the sensor module a signal indicative of a concentration of analyte within the body.

22. The method of claim 21, further including retaining the analyte sensing element below the epidermis for a duration of more than ten days.
